(19) United States
(12) Patent Application Publication
(10) Pub. No.: US 2026/0272433 A1
(43) Pub. Date: Sep. 17, 2026

(54) FELTING-BASED ANCHORING SYSTEMS AND METHODS OF USE THEREOF

(71) Applicant: Edwards Lifesciences Corporation, Irvine, CA (US)

(72) Inventor: Andrew Charles May, Irvine, CA (US)

(21) Appl. No.: 19/673,429

(22) Filed: May 11, 2026

Related U.S. Application Data
(63) Continuation of application No. PCT/US2024/055079, filed on Nov. 8, 2024.
(60) Provisional application No. 63/598,285, filed on Nov. 13, 2023.

(51) Int. Cl. A61B 17/00; A61F 2/24

(52) U.S. Cl. CPC A61B 17/00234; A61F 2/2427; A61F 2/2457; A61F 2/2466; A61B 2017/00243; A61B 2017/003; A61B 2017/00349; A61B 2017/00367; A61F 2220/0008

(57) ABSTRACT

Apparatus for use with tissue of a heart of a subject, the apparatus comprising a textile, a tether, and a tool. The tether extends through the textile. The tool is transluminally advanceable into the heart of the subject, and comprises, at a distal end thereof, multiple barbs. While the textile contacts the tissue, the multiple barbs are adapted to fix the textile to the tissue by repeatedly (i) moving distally through the textile and into the tissue to draw fibers of the textile into the tissue; and (ii) moving proximally out of the tissue and the textile, while leaving the fibers remaining within the tissue. Other embodiments are also described.

### CROSS-REFERENCES TO RELATED APPLICATIONS

The present application is a Continuation of International Patent Application PCT/US2024/055079, filed on Nov. 8, 2024, and titled “Felting-based anchoring systems and methods of use thereof,” which published as WO 2025/106344, and which claims the benefit of Provisional U.S. Patent Application 63/598,285, filed on Nov. 13, 2023. Each of the above disclosures are incorporated by reference for all purposes.

### BACKGROUND

A function of mammalian hearts is based on heart muscle contracting and creating pressure within ventricles of the heart, causing the blood to flow from the ventricles into arteries. Often, when the mammalian heart malfunctions, it can be repaired by insertion of various implants into the heart. Some such implants are anchored to tissue of the heart using tissue anchors having a head and a tissue-engaging element, for example in the shape of a helix or a dart.

Anchoring of implants to tissue using conventional tissue anchors has several limits or disadvantages. For example, the implant is excessively sized, and has excess components, because of the presence of metallic anchoring elements. Additionally, conventional tissue anchors provide a localized load to the tissue, e.g., based on anchor spacing. That localized load may exceed a limit of the tissue, or conversely may require large tissue anchors in order to distribute the load. Furthermore, some anatomical locations cannot tolerate the depth at which a tissue anchor extends into the tissue, and require surface-only adhesion. Such surface-only adhesion is difficult, if not impossible, to attain using tissue anchors.

A procedure in which an implant is anchored to the tissue is a transcatheter annuloplasty procedure, used to ensure that heart valves do not leak and that the leaflets thereof coapt properly. Such annuloplasty procedures can include (i) securing a tether in an arc around the valve being treated by anchoring multiple tissue anchors to the tissue of the annulus in a series around the valve, and then (ii) contracting the annulus by tensioning the tether, to which the anchors are slidably coupled. As such, multiple tissue anchors are required in annuloplasty procedures, suffering from the disadvantages described above.

There is thus a need for improved techniques and devices for securing implants to tissue of the heart, at the surface level thereof, and without requiring driving tissue anchors into the depth of the tissue. There is further a need for improved techniques and devices for carrying out annuloplasty procedures, without requiring use of tissue anchors.

### SUMMARY

This summary is meant to provide some examples and is not intended to be limiting of the scope of the disclosure in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

In accordance with some implementations herein a felting-based anchoring system and/or apparatus usable and/or for use within the heart of a subject (e.g., a living subject, a simulation, etc.). The system/apparatus includes a textile, which includes multiple fibers, and has a tether running longitudinally through the material. The textile is placed in contact with a surface of tissue of the heart of the subject. Multiple barbs, together forming a felting tool, repeatedly enter the tissue through the textile and are extracted from the tissue. Entry of the barbs through the textile into the tissue draws some fibers of the textile into the tissue, which fibers remain within the tissue when the barbs are extracted, thereby attaching the textile to the tissue at the surface level of the tissue. Following attachment of the textile to the tissue, the tether extending through the textile can be tensioned, for example as part of an annuloplasty procedure, to contract the textile attached to the tissue and thus to deform the tissue as necessary.

There is therefore provided, in accordance with some implementations, a system and/or apparatus usable and/or for use with a real or simulated heart of a real or simulated subject, the system/apparatus including a textile, a tether, and/or a felting tool that is transluminally advanceable into the heart of the subject. In some implementations, the tether can extend through the textile (e.g., can be woven along the textile).

In some implementations, the felting tool can have, at a distal end thereof, multiple barbs. For some implementations, when the textile contacts the surface of the tissue, the multiple barbs are adapted to fix the textile to the tissue by repeatedly: moving distally through the textile and into the tissue to draw fibers of the textile into the tissue; and/or moving proximally out of the tissue and the textile while leaving the fibers remaining within the tissue.

For some implementations, the textile comprises a cell-growth-promoting substance.

For some implementations, the textile comprises a synthetic polymer.

For some implementations, the textile comprises a natural polymer.

For some implementations, the textile comprises a combination of multiple different fiber types.

For some implementations, the textile comprises collagen. For some implementations, the textile comprises cellulose. For some implementations, the textile comprises chitin. For some implementations, the textile comprises chitosan. For some implementations, the textile comprises silk. For some implementations, the textile comprises a hydrogel.

For some implementations, the textile comprises polytetrafluoroethylene. For some implementations, wherein the textile comprises polypropylene. For some implementations, the textile is a woven textile. For some implementations, the textile is a woven textile having a weft and a warp, the weft comprising a first fiber type and the warp comprising a second fiber type.

For some implementations, the textile incorporates a radiopaque material.

For some implementations, the textile is a mat of felted fibers.

For some implementations, the textile is a non-woven textile.

For some implementations, the textile is a felt.

For some implementations, the felting tool is sterile.

For some implementations, the textile is sterile.

For some implementations, the felting tool is adapted to position the textile on the tissue.

For some implementations, the multiple barbs of the felting tool include multiple notched barbs.

For some implementations, each of the barbs has multiple notches defined therein.

For some implementations, the notches are configured to catch onto the fibers.

For some implementations, the multiple barbs are of equal length to each other.

For some implementations, the multiple barbs have different lengths to each other.

For some implementations, the felting tool is configured to extend and retract all of the barbs synchronously with each other.

For some implementations, the felting tool is configured to extend and retract the multiple barbs in a staggered manner.

For some implementations, the felting tool is configured such that the repeated extending and retracting of the barbs comprises moving the barbs axially in a reciprocating manner.

For some implementations, the apparatus is configured such that the reciprocating axial movement of the barbs entwines the fibers with the tissue and with each other.

For some implementations, the system further includes a first longitudinal catheter configured to be transluminally advanced toward an anatomical site of the subject, the first longitudinal catheter having a proximal part and a steerable distal part, and a longitudinal axis therebetween, and the textile is advanceable into the heart of the subject via the first longitudinal catheter.

For some implementations, the felting tool is advanceable into the heart of the subject via the first longitudinal catheter.

For some implementations, the system further includes a second longitudinal catheter configured to be transluminally advanced toward an anatomical site of the subject, the second longitudinal catheter having a proximal part and a steerable distal part, and a longitudinal axis therebetween, and the felting tool is advanceable into the heart of the subject via the second longitudinal catheter.

For some implementations, the textile and the felting tool are advanced into the heart of the subject concurrently, such that as a portion of the textile comes into contact with the tissue, the barbs fix that portion of the textile to the tissue.

For some implementations, the entire textile comes into contact with the tissue prior to fixing of any portion of the textile to the tissue.

For some implementations, the apparatus further includes at least one tissue anchor adapted to be advanced into the heart of the subject, and to temporarily anchor the textile to the tissue prior to fixing of the textile to the tissue by the felting tool.

For some implementations, the tether is impervious to the barbs of the felting tool as the barbs move distally through the textile.

For some implementations, the barbs engage and fix to the tissue a first longitudinal segment of the textile, and the tether extends through a second longitudinal segment of the textile, the second longitudinal segment being parallel to and distinct from the first longitudinal segment.

For some implementations, the tether includes a first length of tether extending along a first lateral edge of the textile, and a second length of tether extending along a second lateral edge of the textile, parallel to the first length of tether, such that a longitudinal gap is formed along the textile between the first and second lengths of tether, and the barbs are adapted to fix the textile to the tissue along the longitudinal gap.

For some implementations, the tether extends along a single longitudinal length of the textile, and the felting tool includes a first group of barbs and a second group of barbs, the first and second groups of barbs having a gap therebetween, wherein the barbs are adapted to fix the textile to the tissue along a width of the textile such that the tether is disposed within the gap between the first and second groups of barbs during the fixing.

For some implementations, at least one end of the tether extends outside of the body of the subject, and is accessible for tensioning thereof.

For some implementations, the system further includes a frame, wherein the frame is adapted to hold the textile in position relative to the tissue during operation of the felting tool.

For some implementations, the system further includes at least one implant attached to the textile via the tether, the implant being adapted to be transluminally advanced to the heart of the subject and attached to the tissue by fixing of the textile to the tissue.

For some implementations, longitudinal axes of the multiple barbs are arranged in a plane, such that distal tips of the multiple barbs form a straight line.

For some implementations, multiple barbs are arranged such that distal tips thereof form a ring shape or a circle.

For some implementations, the multiple barbs are arranged around a rotational cam forming part of the felting tool, the rotational cam being associated with a drivebelt, wherein pulling of the drivebelt is adapted to cause the rotational cam to rotate, thereby to drive the multiple barbs into and out of the textile and the tissue.

For some implementations, each of the multiple barbs are attached to the rotational cam by a biasing element, the biasing element adapted to assist in rapid insertion and extraction of the barbs into and out of the textile and the tissue.

For some implementations, pulling of the drivebelt is further adapted to feed the textile onto the tissue prior to felting thereof.

For some implementations, the drivebelt is associated with, or is, the tether extending through the textile.

In accordance with some implementations there is provided an apparatus for use with tissue of a subject (e.g., a living subject and/or a simulated subject). The apparatus can comprise a textile transluminally advanceable into the subject, the textile comprising multiple fibers and a felting tool transluminally advanceable into the subject, the felting tool comprising, at a distal end thereof, one or more barbs. In some implementations, while the textile contacts the tissue, the one or more barbs are adapted to fix the textile to the tissue by repeatedly moving distally through the textile and into the tissue to draw fibers of the textile into the tissue and moving proximally out of the tissue and the textile, while leaving the fibers remaining within the tissue.

In accordance with some implementations there is provided a method usable and/or for use with a real or simulated heart of a real or simulated subject, the method including (i) advancing into the heart of the subject a textile having a tether extending therethrough; (ii) contacting the textile with a surface of tissue of the heart of the subject; (iii) advancing into the heart of the subject a felting tool terminating, at a distal end thereof, in a multiple barbs; (iv) fixing the textile to the tissue by moving a felting tool across the textile while the multiple barbs move in a reciprocating manner (i) distally through the textile and into the tissue in a manner that pushes fibers of the textile into the tissue; and (ii) proximally out of the tissue and the textile, the fibers remaining within the tissue; and/or (v) following the fixing of the textile, tensioning the tether.

For some implementations, the method further comprises applying intermittent pressure to the felted tissue during the fixing step.

For some implementations, the method further comprises applying a pH-adjusted solution to the textile during the fixing step.

For some implementations, the method further comprises applying a composition of mesenchymal stem cells to the tissue following fixing of the textile, the textile providing a scaffold for the mesenchymal stem cells to grow through.

For some implementations, the textile is a non-woven textile, and the advancing of the textile comprises advancing the non-woven textile into the heart of the subject.

For some implementations, the textile is a non-woven textile, and/or the advancing of the textile comprises advancing the non-woven textile into the heart of the subject.

For some implementations, the non-woven textile is a felt, and/or the advancing of the non-woven textile comprises advancing the felt into the heart of the subject.

For some implementations, the felting tool is sterile.

For some implementations, the textile is sterile.

For some implementations, the advancing of the textile includes advancing the textile with the tether extending longitudinally through the textile.

For some implementations, the tensioning of the tether contracts the textile.

For some implementations, advancing of the textile includes advancing the textile distally out of or distally through a first longitudinal catheter.

For some implementations, advancing of the felting tool includes advancing the felting tool distally out of or distally through the first longitudinal catheter.

For some implementations, advancing of felting tool includes advancing the felting tool distally out of or distally through a second longitudinal catheter.

For some implementations, the textile includes a first portion and a second portion, wherein the contacting and the fixing of the first portion of the textile occur prior to the contacting and the fixing of the second portion of the textile.

For some implementations, the contacting of the textile with the surface of the tissue occurs prior to the fixing of any portion of the textile to the tissue.

For some implementations, the method further includes, prior to fixing of the textile to the tissue, anchoring the textile to the tissue using at least one tissue anchor.

For some implementations, the method further includes, following the fixing of the textile to the tissue, removing the at least one tissue anchor.

For some implementations, fixing of the textile to the tissue includes fixing the textile such that the tether is not damaged by the barbs.

For some implementations, fixing of the textile to the tissue includes fixing the textile such that the tether is not pushed into the tissue by the barbs.

For some implementations, fixing of the textile to the tissue includes fixing a first longitudinal segment of the textile to the tissue, and the tether extends through a second longitudinal segment of the textile, the second longitudinal segment being parallel to and distinct from the first longitudinal segment.

For some implementations, the tether includes a first length of tether extending along a first lateral edge of the textile, and a second length of tether extending along a second lateral edge of the textile, parallel to the first length of tether, such that a longitudinal gap is formed along the textile between the first and second lengths of tether, and the fixing of the textile includes fixing the textile to the tissue along the longitudinal gap.

For some implementations, the tether extends along a single longitudinal length of the textile, the multiple barbs includes a first group of barbs and a second group of barbs, the first and second groups of barbs having a gap therebetween, and/or fixing of the textile to the tissue includes fixing the textile to the tissue along a width of the textile such that the tether is disposed within the gap between the first and second groups of barbs during the fixing.

For some implementations, an end of the tether extends outside of the body of the subject, and the tensioning includes pulling on the end of the tether outside the body of the subject.

For some implementations, the method further includes advancing a frame into the heart of the subject, and contacting the textile with the tissue includes placing the textile on the frame, such that the textile is held in position relative to and in contact with the tissue.

For some implementations, the textile has an implant attached thereto via the tether, the method further including transluminally advancing the implant into the heart of the subject, and fixing of the textile to the tissue results in fixing of the implant to the heart of the subject.

For some implementations, the fixing of the textile to the tissue includes pulling a drivebelt associated with a rotational cam having the multiple barbs mounted thereon, thereby causing rotation of the rotational cam and causing the multiple barbs to move in a reciprocating manner through the textile and into the tissue and proximally out of the tissue and the textile.

There is further provided, in accordance with some implementations, an apparatus for use with tissue of a subject, the apparatus including: a textile transluminally advanceable into the subject, the textile including multiple fibers; and/or a felting tool transluminally advanceable into the subject, the felting tool including, at a distal end thereof, one or more barbs, wherein, while the textile contacts the tissue, the one or more barbs are adapted to fix the textile to the tissue by repeatedly: (i) moving distally through the textile and into the tissue to draw fibers of the textile into the tissue; and/or (ii) moving proximally out of the tissue and the textile, while leaving the fibers remaining within the tissue.

For some implementations, the textile is a woven textile.

For some implementations, the textile includes a cell-growth-promoting substance.

For some implementations, the textile includes a synthetic polymer.

For some implementations, the textile includes a natural polymer.

For some implementations, the textile includes a combination of multiple different fiber types.

For some implementations, the textile includes collagen.

For some implementations, the textile includes cellulose.

For some implementations, the textile includes chitin.

For some implementations, the textile includes chitosan.

For some implementations, the textile includes silk.

For some implementations, the textile includes a hydrogel.

For some implementations, the textile includes polytetrafluoroethylene.

For some implementations, the textile includes polypropylene.

For some implementations, the textile is a woven textile.

For some implementations, the textile is a woven textile having a weft and a warp, the weft including a first fiber type and the warp including a second fiber type.

For some implementations, the textile incorporates a radiopaque material.

For some implementations, the textile is a non-woven textile.

For some implementations, the textile is a felt.

For some implementations, the felting tool is sterile.

For some implementations, the textile is sterile.

For some implementations, the felting tool is adapted to position the textile on the tissue.

For some implementations, the one or more barbs of the felting tool each include one or more notches.

For some implementations, the system further includes a first longitudinal catheter configured to be transluminally advanced toward an anatomical site of the subject, the first longitudinal catheter having a proximal part and a steerable distal part, and the textile is advanceable into the heart of the subject via the first longitudinal catheter.

For some implementations, the felting tool is advanceable into the heart of the subject via the first longitudinal catheter.

For some implementations, the system further includes a second longitudinal catheter configured to be transluminally advanced toward an anatomical site of the subject, the second longitudinal catheter having a proximal part and a steerable distal part, and the felting tool is advanceable into the heart of the subject via the second longitudinal catheter.

For some implementations, the textile and the felting tool are advanced into the heart of the subject concurrently, such that as a portion of the textile comes into contact with the tissue, the one or more barbs fix the portion of the textile to the tissue.

For some implementations, the entire textile comes into contact with the tissue prior to fixing of any portion of the textile to the tissue.

For some implementations, the apparatus further includes at least one tissue anchor adapted to be advanced into the heart of the subject, and to temporarily anchor the textile to the tissue prior to fixing of the textile to the tissue by the felting tool.

For some implementations, the system further includes a tether, wherein the barbs engage and fix to the tissue a first longitudinal segment of the textile, and the tether extends through a second longitudinal segment of the textile, the second longitudinal segment being parallel to and distinct from the first longitudinal segment.

For some implementations, the tether includes a first length of tether extending along a first lateral edge of the textile, and a second length of tether extending along a second lateral edge of the textile, parallel to the first length of tether, such that a longitudinal gap is formed along the textile between the first and second lengths of tether, and the barbs are adapted to fix the textile to the tissue along the longitudinal gap.

For some implementations, the apparatus further includes a tether, wherein the tether extends along a single longitudinal length of the textile, and the felting tool includes a first group of barbs and a second group of barbs, the first and second groups of barbs having a gap therebetween, wherein the barbs are adapted to fix the textile to the tissue along a width of the textile such that the tether is disposed within the gap between the first and second groups of barbs during the fixing

For some implementations, the system further includes a tether, wherein at least one end of the tether extends outside of the subject, and is accessible for tensioning thereof.

For some implementations, the system further includes a frame, wherein the frame is adapted to hold the textile in position relative to the tissue during operation of the felting tool.

For some implementations, the system further includes a tether and at least one implant attached to the textile via the tether, the implant being adapted to be transluminally advanced to the heart of the subject and attached to the tissue by fixing of the textile to the tissue.

For some implementations, each of the one or more barbs defines a longitudinal axis, the longitudinal axes of the one or more barbs being arranged in a plane, such that distal tips of the one or more barbs form a straight line.

For some implementations, the one or more barbs are arranged such that distal tips thereof form a ring shape or a circle.

For some implementations, the felting tool includes a rotational cam associated with a drivebelt; and/or the multiple barbs are disposed along the rotational cam in a manner that pulling the drivebelt rotates the rotational cam, thereby sequentially pushing the barbs through the textile into the tissue as the cam rolls along the textile.

For some implementations, each of the one or more barbs is attached to the rotational cam by a biasing element, the biasing element adapted to assist in rapid insertion and extraction of the one or more barbs into and out of the textile and the tissue.

For some implementations, pulling of the drivebelt is further adapted to feed the textile onto the tissue prior to felting thereof.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

The present disclosure will be more fully understood from the following detailed description of applications thereof, taken together with the drawings, in which:

### BRIEF DESCRIPTION OF THE FIGURES

FIGS. 1A, 1B, and 1C are schematic illustrations of steps of a method of treatment of a valve of the heart of a subject, using a felting-based anchoring apparatus in accordance with some implementations;

FIGS. 2A, 2B, 2C, and 2D are schematic illustrations of steps of a method of treatment of a valve of the heart of a subject, using a felting-based anchoring apparatus in accordance with some implementations;

FIGS. 3A and 3B are schematic illustrations of implants anchored to the heart of a subject using a felting-based anchoring apparatus in accordance with some implementations;

FIGS. 4A, 4B, 4C, and 4D are schematic illustrations of non-limiting arrangements of the felting-based anchoring apparatus, in accordance with some implementations;

FIGS. 5A and 5B are schematic illustrations of a felting tool in accordance with some implementations;

FIGS. 6A, 6B, 7A and 7B are schematic illustrations of exemplary textiles and the felting process; and

FIG. 8 is a schematic illustration of a modified arrangement of fibers in a woven textile.

### DETAILED DESCRIPTION

Principles of felting-based anchoring apparatus and techniques may be better understood with reference to the drawings and the following description.

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features can be omitted or simplified in order not to obscure the disclosure. Additionally, in order to avoid undue clutter from having too many reference numbers and lead lines on a particular drawing, some elements may not be explicitly identified in every drawing that contains that element.

It is to be understood that the scope of the disclosure is not limited in its implementation to the details of construction and the arrangement of the components set forth in the following description or illustrated in the drawings. The disclosure is capable of other implementations or of being practiced or carried out in various ways. Furthermore, it is to be understood that the phraseology and terminology employed in the disclosure is for the purpose of description and should not be regarded as limiting.

For the purposes of this disclosure, the term “subject” relates to any mammal, such as humans. In some implementations, the subject can include a living subject or a simulation (e.g., a cadaver, cadaver heart, simulator, imaginary person, etc.)

For the purposes of this implementation, the term “cardiac tissue” relates to any tissue of the heart, and includes, for example, any wall of the heart and any tissue of any heart valve.

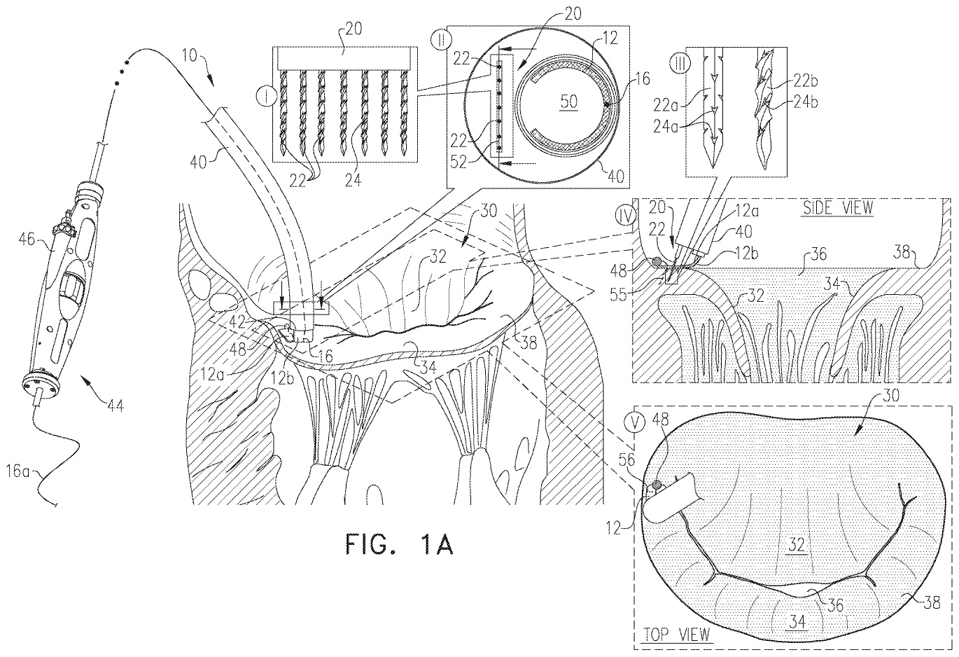

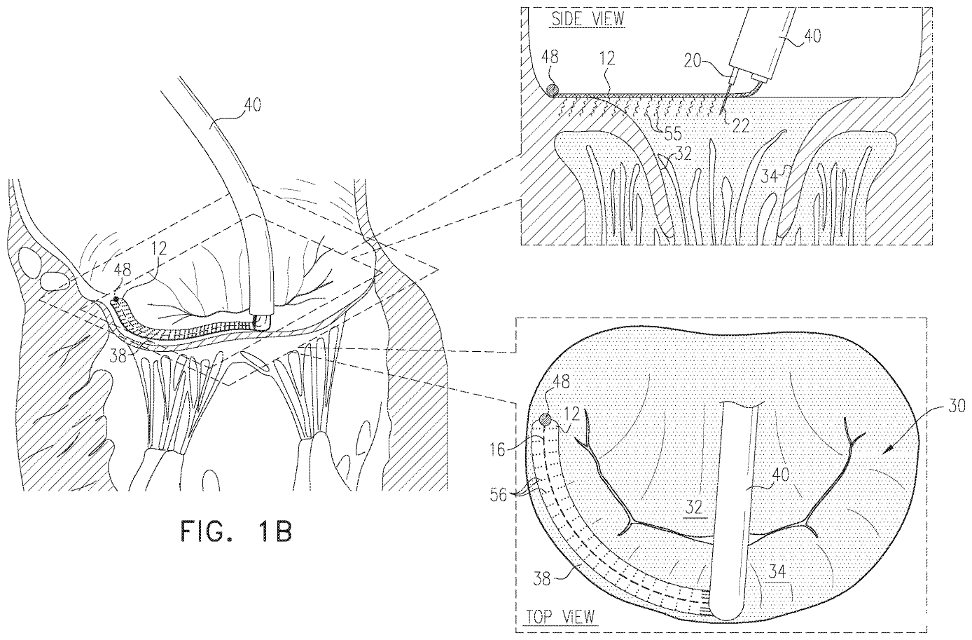

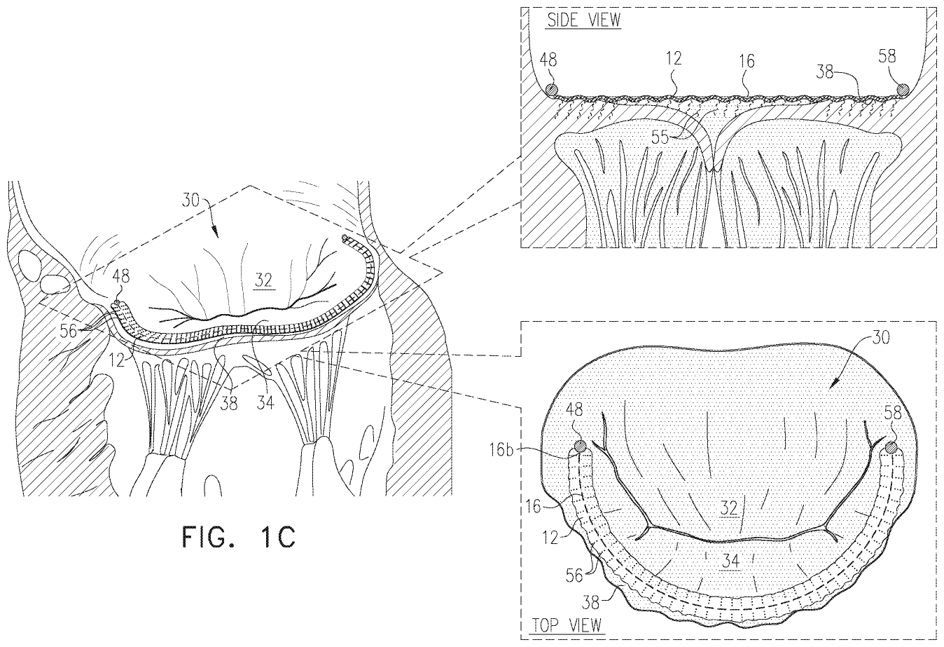

Referring now to the drawings, FIGS. 1A to 1C are schematic illustrations of a method of treatment of a valve of the heart of the subject, using a felting-based anchoring apparatus 10 in accordance with some implementations. Although a mitral valve is illustrated, a skilled artisan will appreciate that this disclosure may be usable and/or used for other valves, including but not limited to the tricuspid valve.

As seen in FIG. 1A, felting-based anchoring apparatus 10 includes textile 12 (e.g., a length or strip of the textile) defining a first broad surface 12a and a second, opposing, broad surface 12b. Textile 12 comprises a multitude of fibers, and has a tether 16 extending longitudinally therethrough. In some implementations, textile 12 can comprise a non-woven fabric such as a felt. Further details and properties of textile 12 are discussed herewithin below. Tether 16 can be formed of any suitable material, such as metal, polymer, biomaterial wire, ribbon, or cord, and has sufficient tensile strength to enable contraction of textile 12 upon tensioning of the tether. For some implementations, textile 12 and tether 16 may be considered to be components of an implant, such as an annuloplasty implant.

In some implementations, the felting-based anchoring apparatus may further comprise a felting tool 20. In some implementations, the felting tool 20 has, at a distal end thereof, one or more barbs 22. In some implementations, the felting tool 20 has multiple barbs 22 (e.g., notched barbs). In some implementations, the barbs 22 can each have multiple notches 24 distributed along a longitudinal length thereof. Box III of FIG. 1A illustrates two non-limiting implementations of notched barbs: notched barb 22a has a triangular shaped needle tip and includes notches 24a directed in a longitudinally distal direction, and notched barb 22b has a spiral contour and includes notches 24b which follow along the direction of the spiral. However, it is to be understood that other variants of notched barbs may be used. For instance, in some implementations, the barbs may comprise a roughened or textured surface. Notches 24a, 24b and/or the roughened or textured surface are configured to catch onto the fibers of the textile.

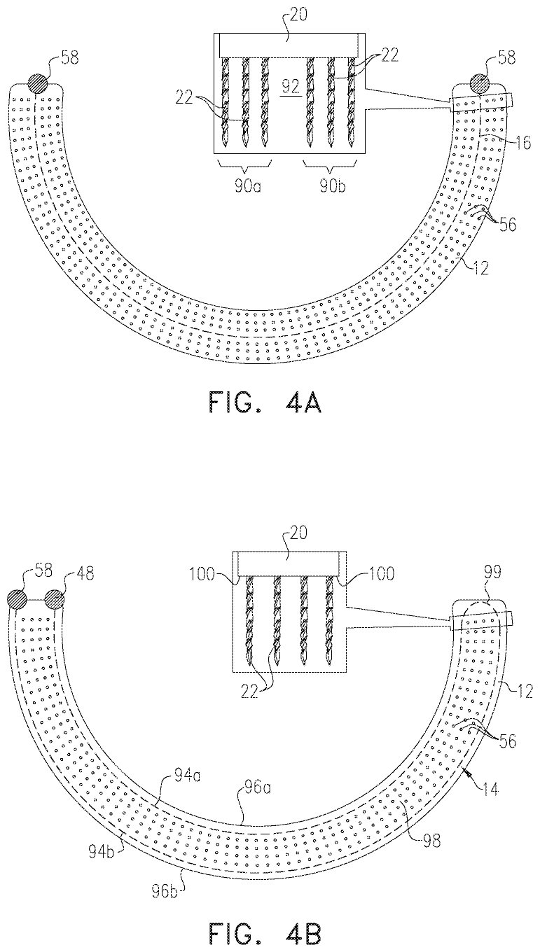

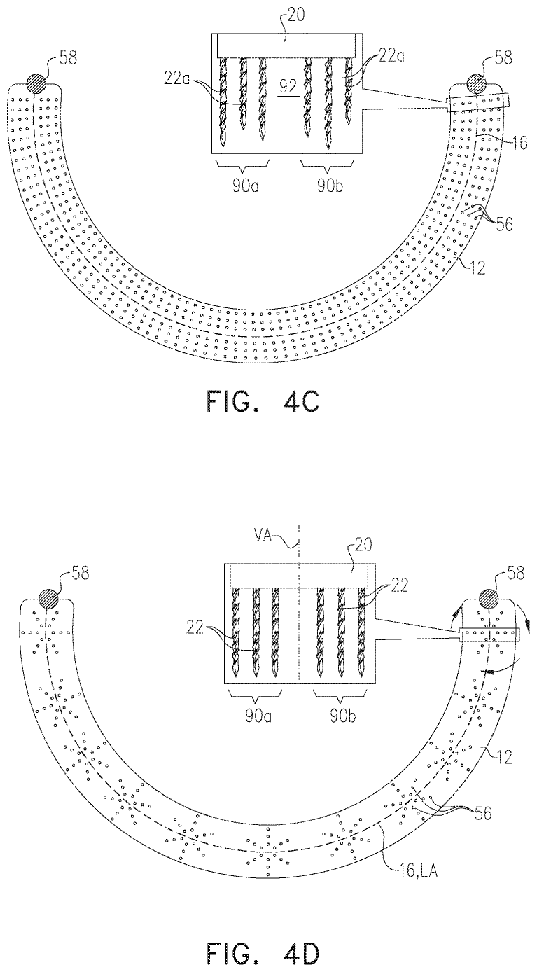

In some implementations, longitudinal axes of notched barbs 22 are arranged in a single plane, such that the distal tips of the notched barbs are arranged a straight line, as shown in Box I of FIG. 1A. In some implementations, distal tips of the notched barbs may be arranged a circle, semi-circle, or a ring shape. In some implementations, notched barbs 22 are arranged in a single plane, as shown in Box I of FIG. 1A. Further non-limiting and combinable examples of barb arrangements are shown in FIGS. 4A-D.

In the illustrated implementation, felting-based anchoring apparatus 10 is used in treatment (e.g., an annuloplasty procedure) of mitral valve 30 of the subject. As seen in Box V of FIG. 1A, leaflets 32 and 34 of the mitral valve do not fully coapt, such that a gap 36 remains therebetween even during ventricular systole. The treatment (e.g., annuloplasty) procedure described herein reshapes the mitral valve 30, such that the leaflets 32 and 34 coapt and gap 36 is reduced or eliminated (see the end result in FIG. 1C). Mitral valve 30 includes an annulus 38, surrounding leaflets 32 and 34.

As seen in FIG. 1A, a longitudinal catheter 40 is advanced into the heart of the subject. In the illustrated implementation, a distal part 42 of the catheter is advanced to an atrium (e.g., a left atrium) of the heart, to be positioned upstream of mitral valve 30. However, it is to be understood that other heart valves (e.g., the tricuspid valve) may be similarly treated. Longitudinal catheter 40 also has an extracorporeal proximal part 44 including a handle 46. Distal part 42 of longitudinal catheter 40 is guidable to the anatomical site, such as by being actively steerable itself (e.g., by being operatively coupled by one or more pullwires to proximal part 44, such as to a steering controller in handle 46), or by being passively guided and/or steered (e.g., by extending over or through another steerable element, such as an actively steerable catheter). A longitudinal axis extends between proximal part 44 and steerable distal part 42 of catheter 40. Catheter 40 can be advanced to the anatomical site using any method known in the art, for example transluminally (e.g., via a vena cava and, if necessary, via the septum separating the atria of the heart) or intercostally.

As shown, textile 12 having tether 16 extending therethrough is advanced to the heart of the subject, for example distally out of longitudinal catheter 40. For some implementations, at least one terminus 16a of tether 16 remains outside of the body of the subject while textile 12 and tether 16 are advanced into the body. In the example shown, a first lock 48 (which may alternatively be referred to as a stopper) is present at a second terminus of tether 16 disposed inside the catheter. In some implementations, the first lock 48 is locked to the tether, securing the second terminus of the tether to textile 12 while the tether is advanced together with the textile into the heart of the subject.

For some implementations, and as illustrated, textile 12 can be advanced to the heart of the subject via a lumen 50 of longitudinal catheter 40, as seen in box II of FIG. 1A. As seen in box IV of FIG. 1A, as a portion of textile 12 is advanced out of longitudinal catheter 40, second broad surface 12b of the textile comes into contact with tissue of the heart of the subject, which, in the illustrated example, is annulus 38.

Similarly, felting tool 20 is advanced to the heart of the subject, for example distally out of longitudinal catheter 40. For some implementations, and as illustrated in box II of FIG. 1A, felting tool 20 can be advanced to the heart of the subject via a dedicated lumen 52 of longitudinal catheter 40. For some implementations, felting tool 20 can be advanced to the heart of the subject together with textile 12 via lumen 50. In additional and/or alternative implementations, felting tool 20 can be advanced to the heart of the subject by a second, dedicated, longitudinal catheter, similar to longitudinal catheter 40.

As seen in box IV of FIG. 1A, as second broad surface 12b of textile 12 is placed in engagement with annulus 38, the textile can be fixed to the annulus by notched barbs 22 of felting tool 20. Specifically, the multiple notched barbs 22 can be repeatedly advanced through textile 12 and into tissue of annulus 38, entering the textile via first broad surface 12a and exiting via second broad surface 12b. During each such advancement, some fibers 55 of textile 12 are snagged on notches 24 of notched barbs 22, and are drawn (e.g., pushed and/or dragged) thereby into the tissue of annulus 38. After each such advancement, the multiple notched barbs 22 are then retraced in a proximal direction out of the tissue and the textile, leaving fibers 55 within the tissue. Fibers 55 extending into annulus 38 form a multitude of fixation points 56, shown in box V of FIG. 1A, for fixing the portion of the textile to the annulus. For the purpose of the illustration, fixation points 56 are shown as discrete points with clear spaces therebetween. However, it is to be understood that the number and proximity of the fixation points may be such that they are indiscrete from each other. For example, needles 22 may, when in operation, repeatedly and rapidly extend and retract in a reciprocating manner.

As explained in detail hereinbelow with respect to FIGS. 4A and 4B, tether 16 and felting tool 20 are configured such that the tether is not damaged, or pushed into annulus 38, by notched barbs 22.

Turning to FIG. 1B, it is seen that catheter 40 gradually feeds out textile 12, and lies the textile along annulus 38. As textile 12 is progressively placed along annulus 38, felting tool 20 progressively fixes the textile to the annulus as described hereinabove. As shown, apparatus 10 may be configured such that, as textile 12 is fed out, it lies across the face of tool 20, for needles 22 to access. This may additionally and/or alternatively be described as apparatus 10 being configured to slide or wipe tool 20 across textile 12 as the textile is fed out,—e.g., with needles 22 continuously in operation.

Fixing of textile 12 to annulus 38 continues until it has been determined that the textile extends along a sufficiently long stretch of the annulus—e.g., sufficiently circumscribes the valve.

FIG. 1C illustrates the final state of mitral valve 30, following termination of the annuloplasty procedure using apparatus 10. As seen, textile 12 circumscribes at least half of annulus 38, and has been fixed to the annulus by felting tool 20.

As seen, the tension in tether 16 has been modified (e.g., tension is applied to the tether), e.g., by pulling on terminus 16a of the tether extending out of the body. Modification of the tension in tether 16 longitudinally contracts textile 12, and thereby the annulus of valve 30, reshaping the valve, so as to close the gap 36. As shown in FIGS. 1C, the reshaping of the mitral valve improves coaptation between leaflets 32 and 34 of the valve, thereby reducing (e.g., closing) gap 36.

Following modification to the tension of the tether and reshaping of the valve, the tension in the tether is locked. In the implementation illustrated in FIGS. 1A to 1C, a first lock 48 (which may alternatively be referred to as a stopper) was present at terminus 16b of tether 16, and locked to the tether, when the tether was initially introduced into the heart of the subject. In some implementations, a second lock 58 is slid along tether 16 (e.g., from terminus 16a disposed outside the subject's body), and is locked to the tether. Locks 48 and 58 can lock the tension in tether 16 by inhibiting the tether from sliding out of textile 12—e.g., by the locks abutting the textile. For some implementations, locks 48 and/or 58 have one or more features described in U.S. patent application Ser. No. 16/534,875 to Brauon et al., filed Aug. 7, 2019, which published as US 2020/0015971, which is incorporated herein by reference.

For some implementations, the textile 12 is predetermined—e.g., a textile of appropriate length is selected for use. For some implementations, textile 12 is secured along a desired stretch of the tissue and excess textile is then trimmed. For some implementations, apparatus 10 may be capable of securing multiple stretches of textile 12 along multiple stretches of tissue without the apparatus being withdrawn from the subject.

For some implementations, textile 12 and/or tool 20 is configured such that, immediately upon felting, it remains possible to remove the textile from the tissue—e.g., the nature and quantity of fibers 55 is such that the textile may be pulled away from the tissue. This may advantageously allow for repositioning if required. For such implementations, after a duration (e.g., of minutes, hours, or days), fibers 55 become more securely fixed to the tissue (e.g., due to tissue growth), so contraction of textile 12 may be deferred until after that duration.

In some implementations, following felting of textile 12, excess tether is cut and removed from the atrium. The advancement of locks 48 and 58, and the cutting of excess tether 16, may be performed using tools known in the art.

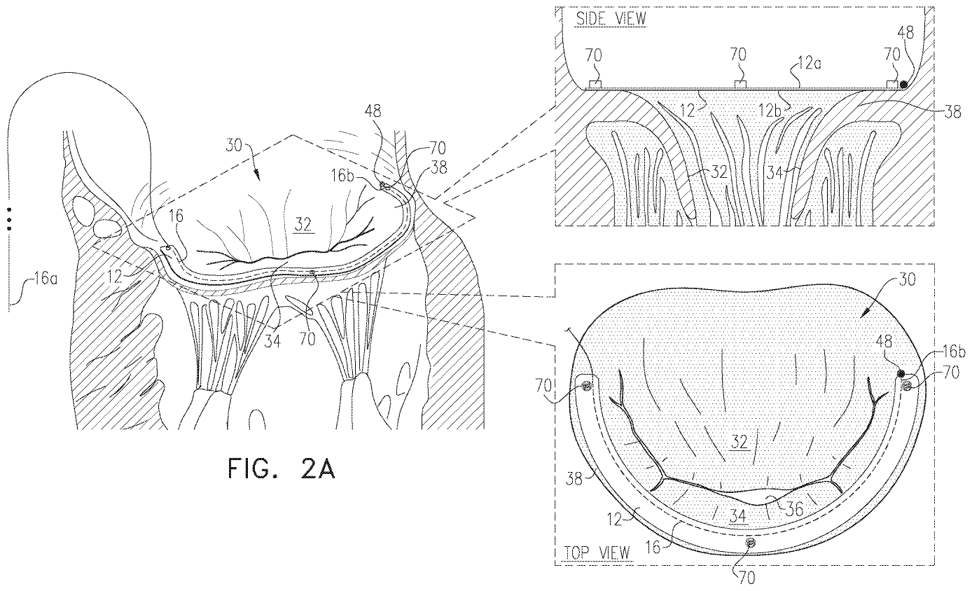

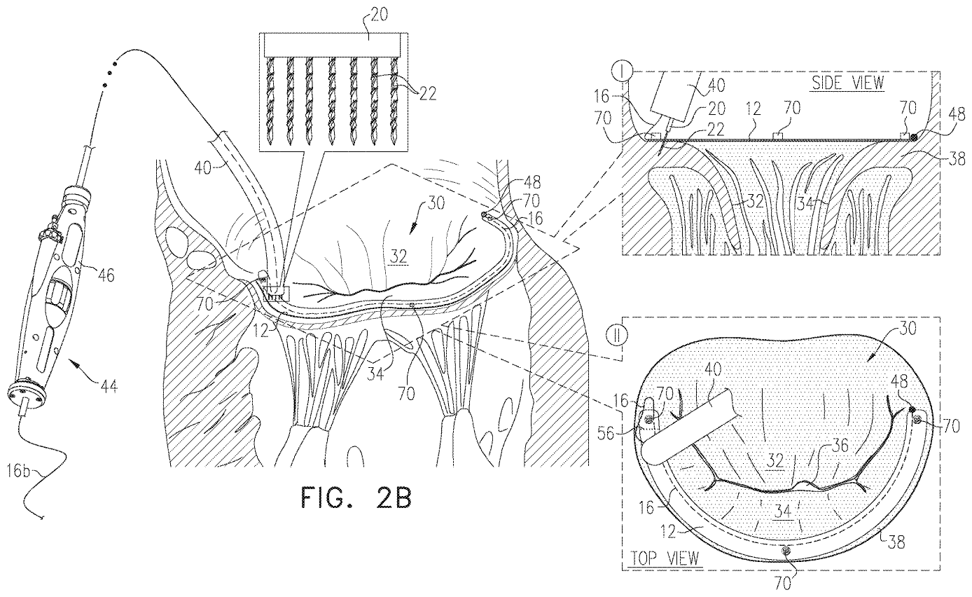

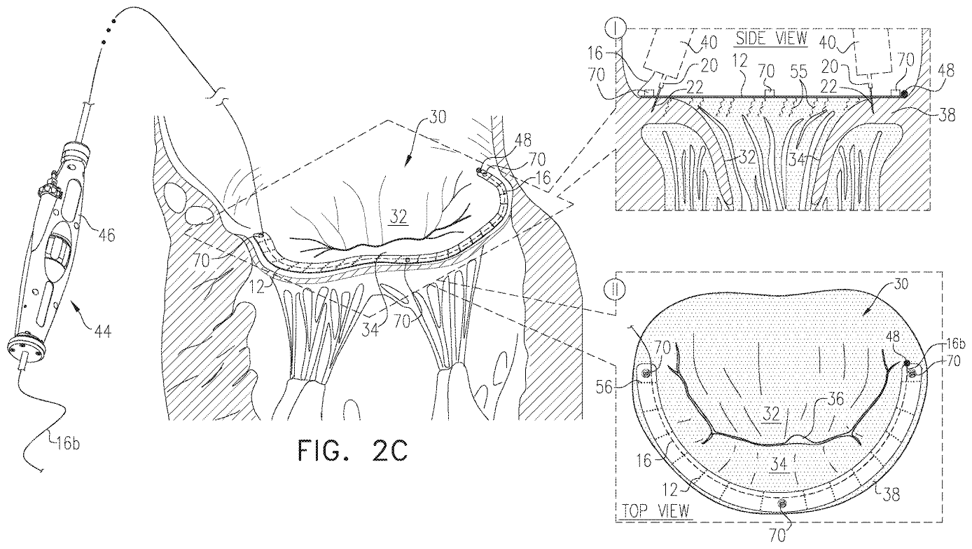

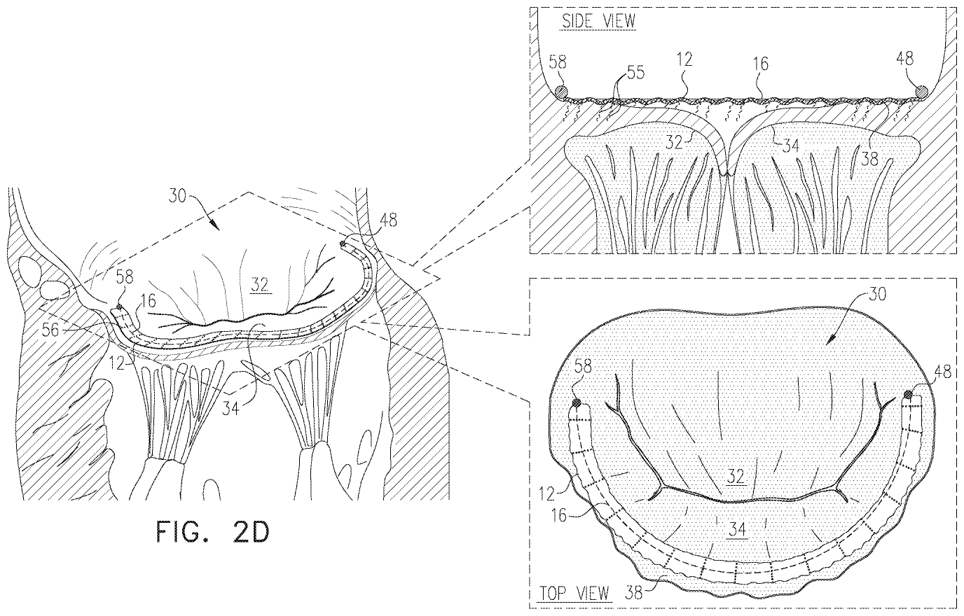

Referring now to the drawings, FIGS. 2A to 2D are schematic illustrations of steps of a method of treatment of a valve of the heart of a subject, using the felting-based anchoring apparatus 10 described hereinabove with respect to FIGS. 1A to 1C, in accordance with some implementations. In some implementations, the subject can include a living subject or a simulation (e.g., a cadaver, cadaver heart, simulator, imaginary person, etc.)

As seen in FIG. 2A, textile 12 is placed within the heart of the subject, along annulus 38 of valve 30. For example, the textile may be advanced to the heart via lumen 50 of longitudinal catheter 40 (FIG. 1A), substantially as described hereinabove with respect to FIGS. 1A to 1C. Textile 12 has tether 16 extending therethrough, with terminus 16a of tether 16 extending outside of the body of the subject while textile 12 and tether 16 are advanced into the body. In some implementations, first lock 48 (which may alternatively be referred to as a stopper) is present at a second terminus 16b of tether 16 and is locked to the tether, while the tether is advanced together with textile 12 into the heart of the subject.

As seen, second broad surface 12b of textile 12 is placed in contact with tissue of the heart of the subject, which, in the illustrated example, is annulus 38. In order to avoid movement of the textile 12 relative to annulus 38 during fixation thereof, temporary tissue anchors 70 may be advanced into the heart of the subject, and driven into the annulus 38 through textile 12, to temporarily anchor the textile to the annulus. As seen in FIG. 2A, tissue anchors 70 are placed such that they do not interfere with tether 16.

For some implementations, tissue anchors 70 are driven into annulus 38 by a driving tool as known in the art. The driving tool can be advanced to the heart of the subject via a second catheter, similar to catheter 40, or via catheter 40. In some implementations, the driving tool is rotatable, and anchors tissue anchors 70 by screwing the tissue anchors through textile 12 and into the tissue of annulus 38. Following anchoring of tissue anchors 70, the driving tool can be retracted from the heart of the subject, for example via the catheter through which the driving tool was introduced. The type of tissue anchor is not intended to limit the scope of the disclosure and other types of tissue anchors, e.g., barbs, darts, etc., may be used.

Turning to FIG. 2B, it is seen that felting tool 20 is advanced to the heart of the subject, for example distally out of longitudinal catheter 40. For some implementations, tether 16 continues to extend out of longitudinal catheter 40 during advancement of felting tool 20 into the heart of the subject. For some implementations, felting tool 20 can be advanced to the heart of the subject by a second, dedicated, longitudinal catheter, similar to longitudinal catheter 40. FIG. 2C shows textile 12 following fixing the entire length thereof to annulus 38 by felting tool 20, such that felting tool 20 is shown in phantom at both ends of the textile. In some embodiments, the entire textile (e.g., a distal surface along the entire length of the textile) may come into contact with the tissue prior to fixing the textile to the tissue.

As seen clearly in box I of FIG. 2B, felting tool 20 starts fixing textile 12 to annulus 38 at one end of the textile, to form fixation points 56. The felting tool 20 can fix textile 12 to annulus 38 is by extension of notched barbs 22 through the textile and into the tissue of the annulus to form fixation points 56, substantially as described hereinabove with respect to FIGS. 1A to 1C. It is to be appreciated that the locations of fixation points 56 are selected such that tissue anchors 70 do not interfere with the fixation process, for example by skipping portions of textile 12 into which the tissue anchors extend.

FIG. 2D illustrates the final state of mitral valve 30, following termination of the annuloplasty procedure using apparatus 10. Textile 12 extends along annulus 38, and has been fixed to the annulus by multiple fixation points 56, generated by felting tool 20. As seen, temporary tissue anchors 70 (FIGS. 2A-2C) have been removed from textile 12. For example, tissue anchors 70 may be removed by rotation of the driving tool in the opposing direction to release the tissue anchors, and retracting of the released tissue anchors through the catheter and out of the body. It is to be understood that, for some implementations, anchors 70 may be left in place, such that they cooperate with the felting to secure textile 12 to the tissue.

As seen in FIG. 2D, the tension in tether 16 has been modified (e.g., tension is applied to the tether) by pulling on terminus 16a of the tether extending out of the body. Modification of the tension in tether 16 causes reshaping of the mitral valve 30 so as to close the gap 36. As shown in FIGS. 2D, the reshaping of the mitral valve improves coaptation between the leaflets thereof, as described hereinabove.

Following modification to the tension of the tether and reshaping of the valve, the tension in the tether is locked, for example by introduction of second lock 58 substantially as described hereinabove. In some implementations, excess tether is then cut and removed from the left atrium. The advancement of locks 48 and 58, and the cutting of excess tether 16, may be performed using tools known in the art.

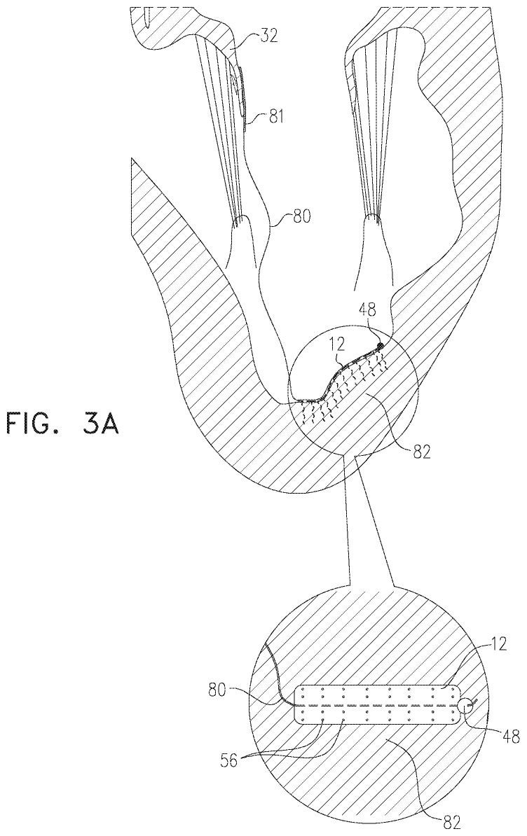

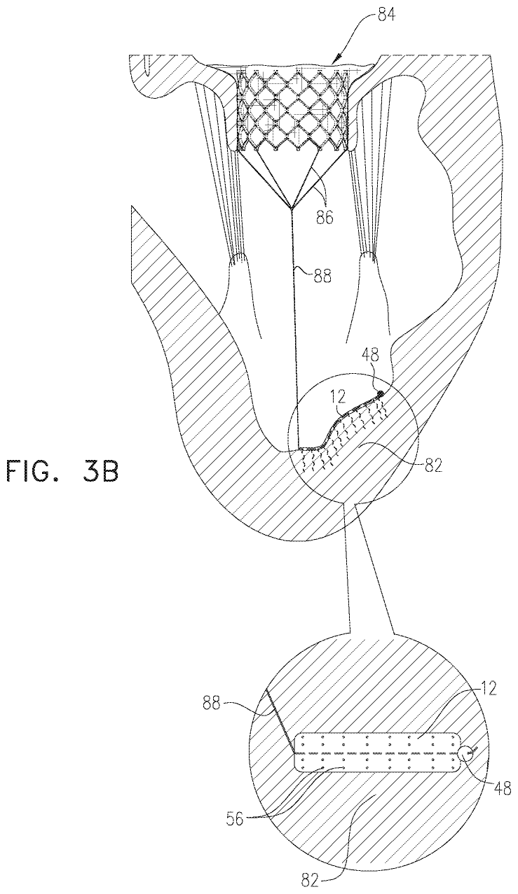

Reference is now made to FIGS. 3A and 3B, which are schematic illustrations of a felt-based anchoring system and/or apparatus anchored to the heart of a subject in accordance with some implementations. In some implementations, the subject can include a living subject or a simulation (e.g., a cadaver, cadaver heart, simulator, imaginary person, etc.)

FIG. 3A illustrates an artificial chord 80 replacing one of the chordae tendineae. A proximal portion of chord 80 is attached to leaflet 32 of the mitral valve (e.g., by a textile 81), and a distal portion of chord 80 extends through textile 12, in a manner similar to that described with respect to tether 16 hereinabove. A lock 48, disposed at the distal end of artificial chord 80, locks the chord to textile 12. As seen, textile 12 has been fixed to the heart muscle 82 by felting, substantially as described hereinabove with respect to FIGS. 1A to 2D, such that multiple fixation points 56 extend along textile 12. In some implementations, the length of chord 80, within the heart, can be controlled by the exact placement of lock 48.

FIG. 3B illustrates an artificial heart valve 84 disposed between leaflets 32 and 34 of the mitral valve of the heart of a user. Artificial heart valve 84 includes multiple tethers 86 merging into a single tether 88, extending distally into the heart of the user. A distal portion of tether 88 extends through textile 12, in a manner similar to that described with respect to tether 16 hereinabove. A lock 48, disposed at the distal end of tether 88, locks the tether to textile 12. As seen, textile 12 has been fixed to the heart muscle 82 by felting, substantially as described hereinabove with respect to FIGS. 1A to 2D, such that multiple fixation points 56 extend along textile 12. This in turn prevents the artificial heart valve 84 from moving out of its location between the leaflets, toward the atrium. It is to be understood by ones of skill in the art that the length of tether 88, within the heart, can be controlled by the exact placement of lock 48.

Reference is now made to FIGS. 4A-D, which are schematic illustrations of several non-limiting arrangements of portions of felting-based anchoring apparatus used in, but not limited to, the methods described with respect to FIGS. 1A-1C and 2A-2D, in accordance with some implementations. FIG. 4A illustrates an exemplary arrangement of a first group 90a and a second group 90b of barbs 22 with respect to tether 16. FIG. 4B illustrates an exemplary arrangement of barbs 22 with respect to lengths 94a and 94b of tether 16. FIG. 4C illustrates an alternate configuration of barbs 22a. FIG. 4D illustrates an exemplary placement of barbs 22 with respect to operation of felting tool 20 and the resulting fixation points 56 within the tissue. It is noted that the implementations and options illustrated in FIGS. 4A-D are independent and may be combined with other variations of implementations shown herein in FIGS. 1A-4B.

As seen in FIGS. 4A and 4B, tether 16 (FIG. 4A), and tether 14 (FIG. 4B) do not coincide with fixation points 56, and as such is not trapped or damaged when textile 12 is fixed to the tissue. For example, tether 16 may extend through textile 12 alongside, but separate from, fixation points 56. In some implementations, fixation points 56 generated by notched barbs 22 are disposed along and/or define a longitudinal segment of textile 12, and tether 16 extends alongside the longitudinal segment (e.g., along a second longitudinal segment of the textile)—e.g., parallel with the longitudinal segment along which the fixation points are disposed.

As seen in FIG. 4A, tether 16 is disposed along the midline of textile 12. Felting tool 20 includes a first group 90a and a second group 90b of notched barbs 22, such that a gap 92 is formed between the two groups of barbs. During fixing of textile 12 to the tissue, first group 90a of notched barbs 22 is disposed on one side of tether 16, and second group 90b is disposed on the opposing side, such that the tether is aligned with gap 92. Consequently, when the barbs 22 extend into textile 12, tether 16 remains untouched by the barbs.

In FIG. 4B, tether 14 includes a first length 94a, which extends along a first lateral edge 96a of textile 12, and a second length 94b extending along a second lateral edge 96b of textile 12, with a longitudinal gap 98 disposed between the two lengths of the tether along the length of the textile (e.g., along the midline of the textile). In felting tool 20, the row of barbs 22 is sufficiently short that fixation points 56 are disposed within longitudinal gap 98. For example, barbs 22 can be disposed medially, e.g., leaving gaps 100 at the edges of tool 20.

In the example shown, tether 14 defines a bight 99 at which the tether doubles-back on itself, the bight connecting lengths 94a and 94b—e.g., at or toward one end of textile 12. For some implementations of this, and as shown, a lock 48 is already in place, securing an end of length 94a to textile 12 prior to delivery, length 94b is pulled in order to tension both lengths of the tether and contract the textile, and a lock 58 is subsequently locked to length 94b in order to lock in the tension—e.g., as described hereinabove, mutatis mutandis.

In some implementations, both lengths of tether 14 are pulled in order to tension the tether, and the tension is subsequently locked in by locking a respective lock 58 to each end of the tether, or by locking a single lock 58 to both ends of the tether.

In some implementations, rather than having a single tether 14 double back on itself to define two lengths, two individual tethers are arranged parallel with each other to similar effect.

FIG. 4C shows barbs 22a extended to different lengths. In some implementations, barbs 22a have different lengths to each other. Thus, for such implementations, the different lengths shown in the inset of FIG. 4C may be considered to represent the actual lengths of the barbs. Accordingly, when extended by felting tool 20 through textile 12 into the tissue, the tips penetrate the tissue at different depths. The resulting variable distribution of fibers within the tissue may advantageously strengthen the felting process and connection between the textile and the tissue.

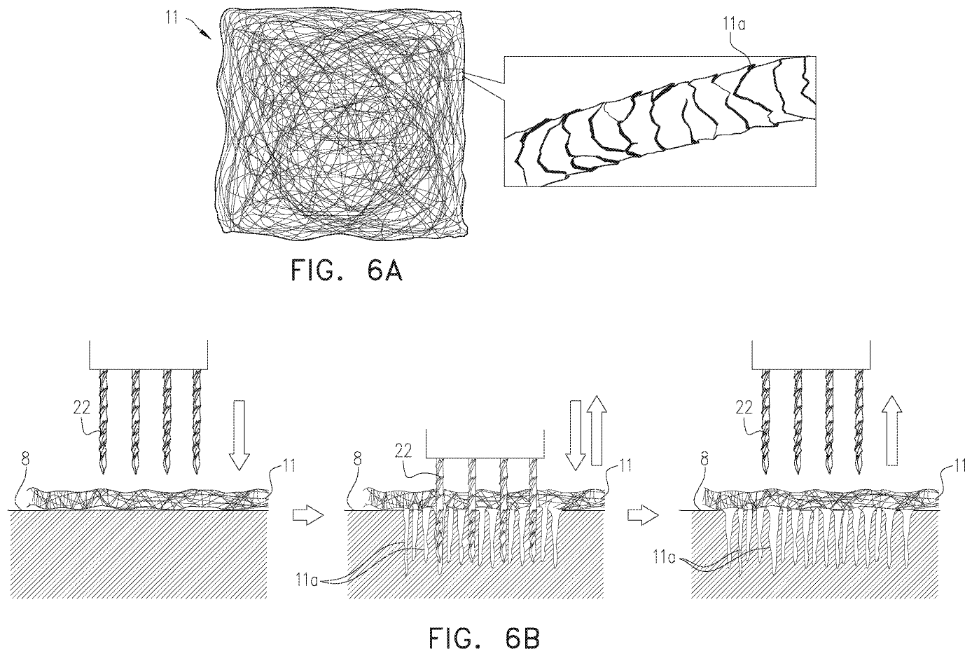

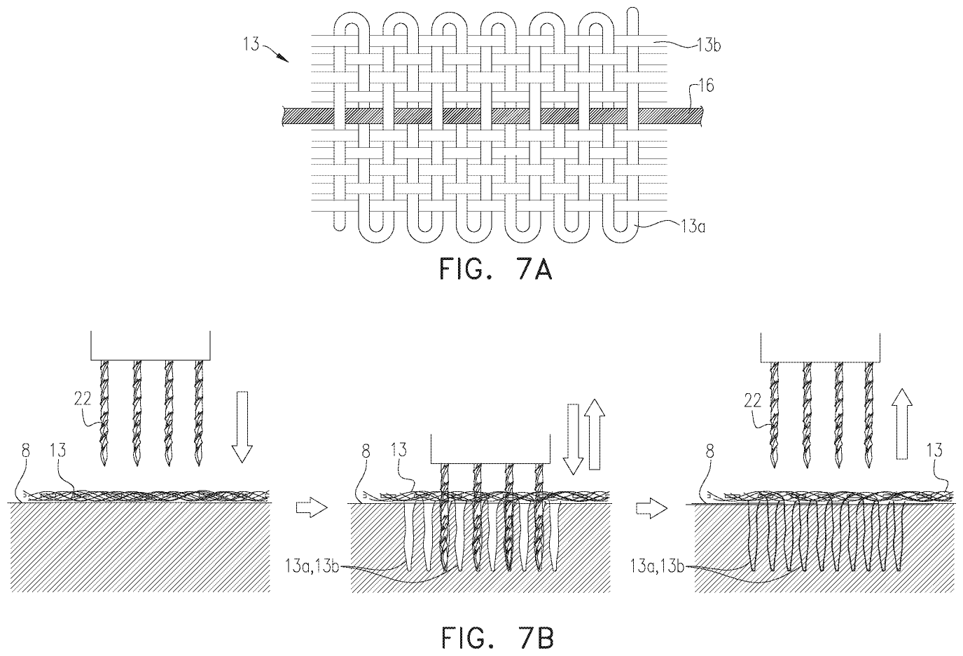

In some implementations, felting tool 20 is configured to extend and retract all of the barbs synchronously with each other. In some implementations, felting tool 20 is configured to extend and retract barbs 22a in a staggered manner (e.g. similar to the movement of pistons in an internal combustion engine). Thus, for such implementations, the inset of FIG. 4C may be considered to represent a snapshot in time. In such implementations, barbs 22a may themselves be of equal length to each other. In some implementations, felting tool 20 is configured such that the repeated extending and retracting of barbs 22 comprises moving the barbs axially in a reciprocating manner. In some implementations, apparatus 10 is configured such that the reciprocating axial movement of the barbs entwines the fibers with the tissue and with each other (as shown in FIGS. 6B and 7B).

In FIG. 4D, felting tool 20 is configured to alternately push barbs 22 through the textile into the tissue at a sequence of different angles around a central, e.g., vertical, axis VA of the multiple barbs 22. Central vertical axis VA may be positioned, e.g., between groups 90a and 90b of barbs 22. Over the course of the felting process, after pushing the barbs into the tissue at multiple different angles along vertical axis VA at a single location along longitudinal axis LA, the felting tool is advanced a step forward along the longitudinal axis. The process is repeated of in a reciprocating manner extending the barbs 22 through the tissue at multiple angles around axis VA while driving the fibers into the tissue. As in FIGS. 4A and 4C, barbs 22 are arranged to avoid penetrating the textile and tissue along the path of the tether 16, even when rotated around axis VA.

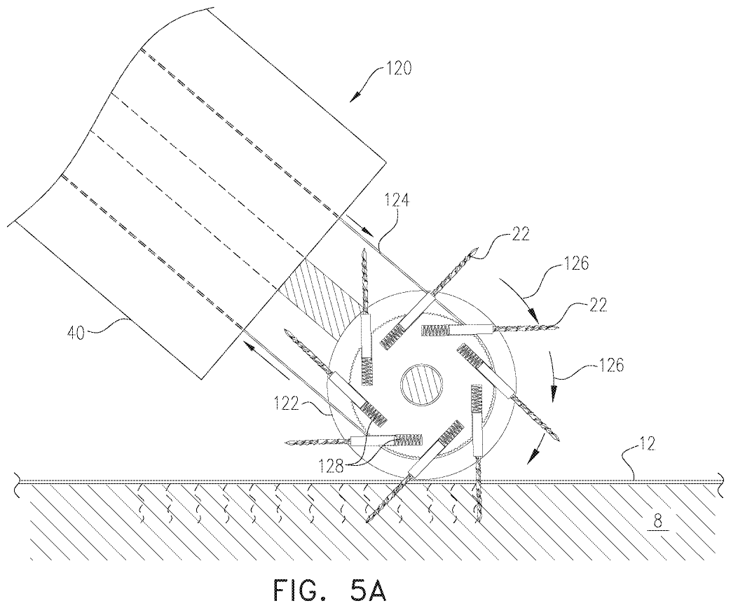

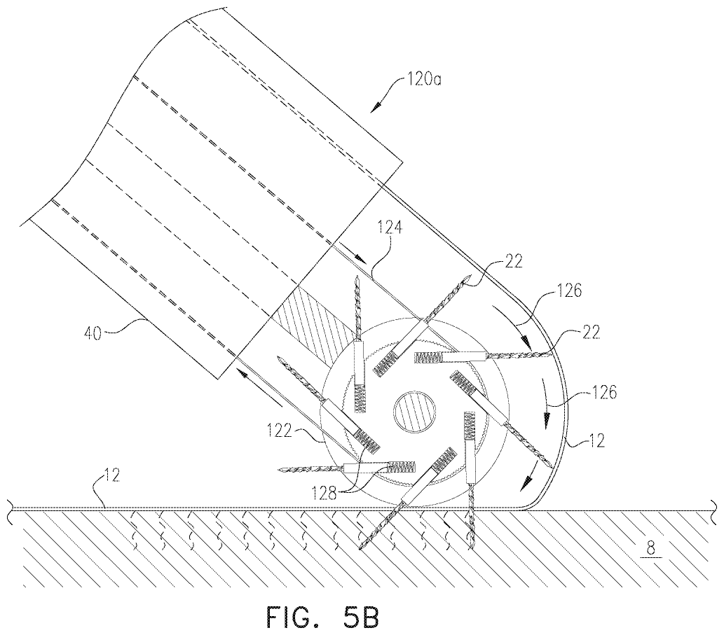

Reference is now made to FIGS. 5A-B, which are schematic illustrations of a felting tool 120, 120a in accordance with some implementations. In FIG. 5A, textile 12 has been applied to a surface of tissue 8 prior to barbs 22 being extended through both textile 12 and tissue 8. In FIG. 5B, textile 12 is applied to the surface of tissue 8 as barbs 22 are rotated along the tissue.

In FIG. 5A, textile 12 is disposed over the surface of tissue 8. Felting tool 120 includes multiple barbs 22 (which may be substantially as described hereinabove, mutatis mutandis) mounted onto a rotational cam 122, e.g., a cylindrical roller, functionally associated with a drivebelt (or drivestring) 124. Pulling of drivebelt 124 causes rotational cam 122 to rotate in the direction of arrows 126, thereby causing barbs 22 to repeatedly enter and exit through textile 12 and the tissue surface and causing felting of the textile, as described hereinabove.

In FIG. 5B, drivebelt 124 is shown associated with textile 12, such that pulling of the drivebelt feeds textile 12 out of a catheter 40 of felting tool 120a and onto the surface of tissue 8, as well as causing barbs 22 to carry out the felting process.

In some implementations, the drivebelt may be associated with, or may actually be, tether 16. In some implementations, each of barbs 22 is associated with a spring or biasing element 128, which assists in the retraction of the barb from tissue 8 and textile 12.

In some implementations, rather than cam 122 being operated/powered from outside of the subject (e.g. via a drivebelt), it is rotated by being rolled along the tissue and/or the textile as the distal end of the felting tool is moved along the tissue. In some such implementations, this rolling draws the textile out of the catheter—e.g. similarly to a tape-based writing correction pen.

It is to be appreciated that use of felting tool 120 shown in FIGS. 5A-B may, in accordance with some implementations, result in more rapid actuation of barbs 22 than linear actuation thereof, as described hereinabove with respect to FIGS. 1A to 2D. For example, this may be true when rotational actuation of the barbs is compared to actuating the barbs via a catheter or other long tube, where each motion at the proximal end of the catheter must be delivered to the distal end for activation of the barbs, and vice versa.

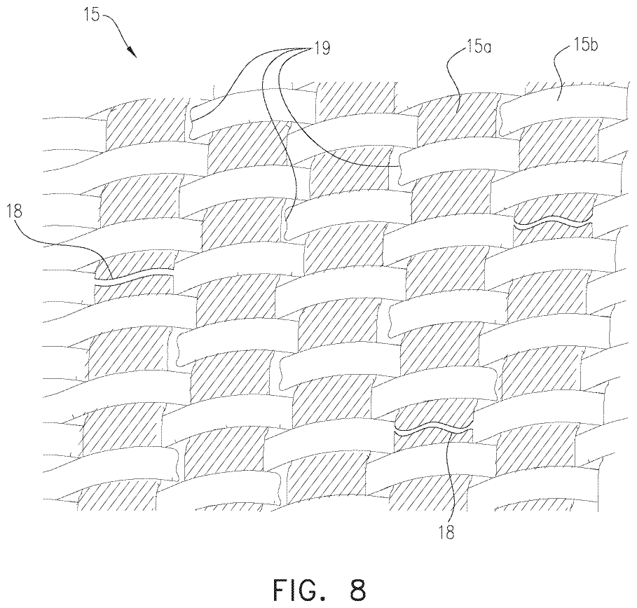

Reference is now made to FIGS. 6A-B, 7A-B, and 8, which are schematic illustrations of several non-limiting examples of arrangements of textile fibers. Textiles 11, 13, and 15 are exemplary variations of textile 12, and may be substituted for textile 12 in any of the implementations discussed in FIGS. 1A-5. FIGS. 6A and 7A show textiles 11 and 13, respectively, which have different arrangements of the fibers from which they are formed. FIGS. 6B and 7B show a process of integrating the textile into the tissue during the felting process. FIG. 8 illustrates an implementation of a woven textile 15 with a modified arrangement of fibers.

In FIG. 6A, textile 11 is shown as a felt-like mass of fibers 11a. Such fibers have a random arrangement, a feature that may contribute to the formation of unravelable knots within the tissue during the felting process shown in FIG. 6B. The box shows an enlargement of a single fiber 11a of the textile, illustrating a rough outer surface. The rough surface of the fibers, especially when brought into contact with notches 24 on barbs 22 by felting tool 20 (see, e.g., FIG. 1A, box III), may contribute to integration of the fibers 11a into the tissue and/or facilitate the formation of a tangle of fibers within the tissue.

In FIG. 7A, textile 13 is shown as a (e.g. loosely) woven fabric-like textile. The textile is comprised of two intertwined layers of fibers at right angles, e.g., a warp 13a and a weft 13b, an arrangement that provides both strength and a pre-felting integration of the two layers. The felting process shown in FIG. 7B creates a felted mass of the fibers in the tissue, with the fibers at right angles contributing significantly to resistance to pull-out forces. Barbs 22 engage and push within tissue 8 fibers 13a of the textile and fibers 13b running perpendicular to and distinct from the fibers 13a.

In FIG. 8, a woven textile 15 similar to that shown in FIG. 7A is shown at higher magnification. Fibers 15a and 15b are woven into a loose fabric. Prior to use in the felting process, the weave is disrupted by creating breaks 18 in warp fibers 15a and/or breaks 19 in weft fibers 15b. The breaks may advantageously enable the barbs to more easily push the fibers out of the plane of the fabric and into the tissue. Breaks may be made in the tissue by, e.g., laser, ionizing radiation, or mechanical means. Similarly, chemical or mechanical treatments may be used to provide fibers with a roughened surface for enhancing integration into tissue 8.

Reference is again made to FIGS. 6A-8. The specific arrangements of fibers in the textile may provide advantages and/or properties that may be particularly beneficial to the integration of the fibers with the tissue, thus contributing to the strength and longevity of the implant in the tissue. Furthermore, structural, surface, and/or chemical features of the textile may provide considerable impact on the success of integration and strength of the textile-tissue bond. For example, the textile fibers may comprise one or more of the following natural or synthetic molecular structures, as non-limiting examples: collagen, cellulose, chitin, chitosan, silk, a hydrogel, polypropylene, polytetrafluoroethylene, a synthetic polymer, and/or a natural polymer.

Various combinations of the above-mentioned fiber types may be combined to enhance the integration and strength of the implant-tissue construct. In some implementations, the textile may comprise different fiber types having different properties. For example, in cases in which the textile comprises a woven textile having a warp and a weft, the warp may comprise a first fiber type and the weft a second fiber type. In some implementations, the textile may incorporate a radiopaque material, e.g., a thread of a radiopaque fiber.

In some implementations, the textile fibers may be treated prior to the implantation procedure to enhance integration (felting) of fibers with each other and/or with the tissue. Such treatment may include, e.g., biochemical treatment or coating of the fibers before/during/after implantation, pH-adjustment, and/or coating with a chemical to stimulate cell growth, tissue repair and/or scar formation. The textile may be configured such that mesenchymal stem cells may be incorporated into/onto the textile, e.g., into a hydrogel, just before implantation.

During the felting process, e.g., as shown in FIGS. 6B and 7B, various additional steps may be included, such as applying intermittent pressure to the tissue, applying a pH-adjusted solution to the textile, and/or applying a solution of mesenchymal stem cells or hydrogel.

The various systems, devices, assemblies, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) such sterilization of the associated system, device, assembly, apparatus, etc. Furthermore, the scope of the present disclosure includes, for some applications, sterilizing one or more of any of the various systems, devices, assemblies, apparatuses, etc. (e.g., with heat, radiation, ethylene oxide, hydrogen peroxide, etc.).

The techniques, methods, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

The present disclosure is not limited to what has been particularly shown and described hereinabove. Rather, the scope of the present disclosure includes both combinations and subcombinations of the various features described hereinabove, as well as variations and modifications thereof that are not in the prior art, which would occur to persons skilled in the art upon reading the foregoing description. Further, the techniques, methods, operations, steps, etc. described or suggested herein can be performed on a living animal or on a non-living simulation, such as on a cadaver, cadaver heart, simulator (e.g., with the body parts, tissue, etc. being simulated), etc.

Although the operations of some of the disclosed implementations are described in a particular, sequential order for convenient presentation, it should be understood that this manner of description encompasses rearrangement, unless a particular ordering is required by specific language set forth above. For example, operations or steps described sequentially can in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms can vary depending on the particular implementation and are discernible by one of ordinary skill in the art.

### Example Applications (Some Non-Limiting Examples of the Concepts Herein are Recited Below)

Example 1. Apparatus for use with tissue of a real or simulated heart of a real or simulated subject, the apparatus comprising a textile, a tether, and/or a felting tool. The textile is configured to extend through the textile. The felting tool is transluminally advanceable into the real or simulated heart of the real or simulated subject. The felting tool comprises, at a distal end thereof, multiple barbs. While the textile contacts the tissue, the felting tool is adapted to secure the textile to the tissue by repeatedly: (i) extending the barbs through the textile and/or into the tissue to draw fibers of the textile into the tissue; and/or (ii) retracting the barbs out of the tissue and/or the textile, while leaving the fibers remaining within the tissue.
Example 2. The apparatus according to example 1, wherein the textile comprises a cell-growth-promoting substance.
Example 3. The apparatus according to any one of examples 1-2, wherein the textile comprises a synthetic polymer.
Example 4. The apparatus according to any one of examples 1-3, wherein the textile comprises a natural polymer.
Example 5. The apparatus according to any one of examples 1-4, wherein the textile comprises a combination of multiple different fiber types.
Example 6. The apparatus according to any one of examples 1-5, wherein the textile comprises collagen.
Example 7. The apparatus according to any one of examples 1-6, wherein the textile comprises cellulose.
Example 8. The apparatus according to any one of examples 1-7, wherein the textile comprises chitin.
Example 9. The apparatus according to any one of examples 1-8, wherein the textile comprises chitosan.
Example 10. The apparatus according to any one of examples 1-9, wherein the textile comprises silk.
Example 11. The apparatus according to any one of examples 1-10, wherein the textile comprises a hydrogel.
Example 12. The apparatus according to any one of examples 1-11, wherein the textile comprises polytetrafluoroethylene.
Example 13. The apparatus according to any one of examples 1-12, wherein the textile comprises polypropylene.
Example 14. The apparatus according to any one of examples 1-13, wherein the textile is a woven textile.
Example 15. The apparatus according to any one of examples 1-14, wherein the textile is a woven textile having a weft and/or a warp, the weft comprising a first fiber type and/or the warp comprising a second fiber type.
Example 16. The apparatus according to any one of examples 1-15, wherein the textile incorporates a radiopaque material.
Example 17. The apparatus according to any one of examples 1-16, wherein the textile is a non-woven textile.
Example 18. The apparatus according to any one of examples 1-17, wherein the textile is a mat of felted fibers.
Example 19. The apparatus according to any one of examples 1-18, wherein the felting tool is sterile.
Example 20. The apparatus according to any one of examples 1-19, wherein the textile is sterile.
Example 21. The apparatus according to any one of examples 1-20, wherein the felting tool is adapted to position the textile on the tissue.
Example 22. The apparatus according to any one of examples 1-21, wherein each of the barbs has multiple notches defined therein.
Example 23. The apparatus according to example 22, wherein the notches are configured to catch onto the fibers.
Example 24. The apparatus according to example 22, wherein the multiple barbs are of equal length to each other.
Example 25. The apparatus according to example 22, wherein the multiple barbs have different lengths to each other.
Example 26. The apparatus according to example 22, wherein the felting tool is configured to extend and/or retract all of the barbs synchronously with each other.
Example 27. The apparatus according to example 22, wherein the felting tool is configured to extend and/or retract the multiple barbs in a staggered manner.
Example 28. The apparatus according to example 22, wherein the felting tool is configured such that the repeated extending and/or retracting of the barbs comprises moving the barbs axially in a reciprocating manner.
Example 29. The apparatus according to example 28, wherein the apparatus is configured such that the reciprocating axial movement of the barbs entwines the fibers with the tissue and/or with each other.
Example 30. The apparatus according to any one of examples 1-29, comprising a first longitudinal catheter configured to be transluminally advanced toward an anatomical site of the subject, the first longitudinal catheter having a proximal part and/or a steerable distal part, and/or wherein the textile is advanceable into the heart of the real or simulated subject via the first longitudinal catheter.
Example 31. The apparatus according to example 30, wherein the felting tool is advanceable into the heart of the real or simulated subject via the first longitudinal catheter.
Example 32. The apparatus according to any one of examples 1-31, further comprising a second longitudinal catheter configured to be transluminally advanced toward an anatomical site of the subject, the second longitudinal catheter having a proximal part and/or a steerable distal part, and/or wherein the felting tool is advanceable into the heart of the real or simulated subject via the second longitudinal catheter.
Example 33. The apparatus according to any one of examples 1-32, wherein the textile and/or the felting tool are advanced into the heart of the real or simulated subject concurrently, such that as a portion of the textile comes into contact with the tissue, the barbs fix the portion of the textile to the tissue.
Example 34. The apparatus according to any one of examples 1-33, wherein the entire textile comes into contact with the tissue prior to fixing of any portion of the textile to the tissue.
Example 35. The apparatus according to example 34, further comprising at least one tissue anchor adapted to be advanced into the heart of the real or simulated subject, and/or to temporarily anchor the textile to the tissue prior to fixing of the textile to the tissue by the felting tool.
Example 36. The apparatus according to any one of examples 1-35, wherein the tether is impervious to the barbs of the felting tool as the barbs move distally through the textile.
Example 37. The apparatus according to any one of examples 1-36, wherein the barbs engage and/or fix to the tissue a first longitudinal segment of the textile, and/or wherein the tether extends through a second longitudinal segment of the textile, the second longitudinal segment being parallel to and/or distinct from the first longitudinal segment.
Example 38. The apparatus according to example 37, wherein the tether includes a first length of tether extending along a first lateral edge of the textile, and/or a second length of tether extending along a second lateral edge of the textile, parallel to the first length of tether, such that a longitudinal gap is formed along the textile between the first and/or second lengths of tether, and/or wherein the barbs are adapted to fix the textile to the tissue along the longitudinal gap.
Example 39. The apparatus according to example 37, wherein the tether extends along a single longitudinal length of the textile, and/or wherein the multiple barbs define a first group of barbs and/or a second group of barbs, the first and/or second groups of barbs having a gap therebetween, wherein the barbs are adapted to fix the textile to the tissue along a width of the textile such that the tether is disposed within the gap between the first and/or second groups of barbs during the fixing.
Example 40. The apparatus according to any one of examples 1-39, wherein at least one end of the tether extends outside of the subject, and/or is accessible for tensioning thereof.
Example 41. The apparatus according to any one of examples 1-40, further comprising a frame, wherein the frame is adapted to hold the textile in position relative to the tissue during operation of the felting tool.
Example 42. The apparatus according to any one of examples 1-41, further comprising at least one implant attached to the textile via the tether, the implant being adapted to be transluminally advanced to the heart of the real or simulated subject and/or attached to the tissue by fixing of the textile to the tissue.
Example 43. The apparatus according to any one of examples 1-42, wherein longitudinal axes of the multiple barbs are arranged in a plane, such that distal tips of the multiple barbs form a straight line.
Example 44. The apparatus according to any one of examples 1-43, wherein the multiple barbs are arranged such that distal tips thereof form a ring shape or a circle.
Example 45. The apparatus according to any one of examples 1-44, wherein: (i) the felting tool comprises a rotational cam associated with a drivebelt; and/or (ii) the multiple barbs are disposed along the rotational cam in a manner that pulling the drivebelt rotates the rotational cam, thereby sequentially pushing the barbs through the textile into the tissue as the cam rolls along the textile.
Example 46. The apparatus according to example 45, wherein each of the multiple barbs is attached to the rotational cam by a biasing element, the biasing element adapted to assist in rapid insertion and/or extraction of the barbs into and/or out of the textile and/or the tissue.
Example 47. The apparatus according to example 45, wherein pulling of the drivebelt is further adapted to feed the textile onto the tissue prior to felting thereof.
Example 48. The apparatus according to example 45, wherein the drivebelt is associated with, or is, the tether extending through the textile.
Example 49. A method for use with tissue of a real or simulated heart of a real or simulated subject, the method comprising: (i) advancing into the heart of the real or simulated subject a textile having a tether extending therethrough; (ii) contacting the textile with a surface of a tissue of the heart of the real or simulated subject; (iii) advancing into the heart of the real or simulated subject a felting tool that includes, at a distal end thereof, multiple barbs; (iv) fixing the textile to the tissue by moving the felting tool across the textile while the multiple barbs move in a reciprocating manner (a) distally through the textile and/or into the tissue in a manner that pushes fibers of the textile into the tissue; and/or (b) proximally out of the tissue and/or the textile, the fibers remaining within the tissue; and/or (v) following fixing of the textile, tensioning the tether.
Example 50. The method according to example 49, further comprising applying intermittent pressure to the felted tissue during the fixing step.
Example 51. The method according to any one of examples 49-50, further comprising applying a pH-adjusted solution to the textile during the fixing step.
Example 52. The method according to any one of examples 49-51, further comprising applying a composition of mesenchymal stem cells to the tissue following fixing of the textile, the textile providing a scaffold for the mesenchymal stem cells to grow through.
Example 53. The method according to any one of examples 49-52, wherein the textile is a non-woven textile, and/or wherein the advancing of the textile comprises advancing the non-woven textile into the heart of the real or simulated subject.
Example 54. The method according to example 53, wherein the non-woven textile is a felt, and/or wherein the advancing of the non-woven textile comprises advancing the felt into the heart of the real or simulated subject.
Example 55. The method according to any one of examples 49-54, further comprising sterilizing the felting tool prior to advancing the felting tool.
Example 56. The method according to any one of examples 49-55, further comprising sterilizing the textile prior to advancing the textile.
Example 57. The method according to any one of examples 49-56, wherein the advancing of the textile comprises advancing the textile with the tether extending longitudinally through the textile.
Example 58. The method according to any one of examples 49-57, wherein the tensioning of the tether contracts the textile.
Example 59. The method according to any one of examples 49-58, wherein advancing of the textile comprises advancing the textile distally out of or distally through a first longitudinal catheter.
Example 60. The method according to example 59, wherein advancing of the felting tool comprises advancing the felting tool distally out of or distally through the first longitudinal catheter.
Example 61. The method according to example 59, wherein advancing of felting tool comprises advancing the felting tool distally out of or distally through a second longitudinal catheter.
Example 62. The method according to any one of examples 49-61, wherein the textile includes a first portion and/or a second portion, wherein the contacting and/or the fixing of the first portion of the textile occur prior to the contacting and/or the fixing of the second portion of the textile.
Example 63. The method according to any one of examples 49-62, wherein the contacting of the textile with the surface of the tissue occurs prior to the fixing of any portion of the textile to the tissue.
Example 64. The method according to example 63, further comprising, prior to fixing of the textile to the tissue, anchoring the textile to the tissue using at least one tissue anchor.
Example 65. The method according to example 64, further comprising, following the fixing of the textile to the tissue, removing the at least one tissue anchor.
Example 66. The method according to any one of examples 49-65, wherein fixing of the textile to the tissue comprises fixing the textile such that the tether is not damaged by the barbs.
Example 67. The method according to any one of examples 49-66, wherein fixing of the textile to the tissue comprises fixing the textile such that the tether is not pushed into the tissue by the barbs.
Example 68. The method according to any one of examples 49-67, wherein fixing of the textile to the tissue comprises fixing a first longitudinal segment of the textile to the tissue, and/or wherein the tether extends through a second longitudinal segment of the textile, the second longitudinal segment being parallel to and/or distinct from the first longitudinal segment.
Example 69. The method according to example 68, wherein the tether includes a first length of tether extending along a first lateral edge of the textile, and/or a second length of tether extending along a second lateral edge of the textile, parallel to the first length of tether, such that a longitudinal gap is formed along the textile between the first and second lengths of tether, and/or wherein the fixing of the textile comprises fixing the textile to the tissue along the longitudinal gap.
Example 70. The method according to example 68, wherein: (i) the tether extends along a single longitudinal length of the textile, (ii) the multiple barbs includes a first group of barbs and/or a second group of barbs, the first and/or second groups of barbs having a gap therebetween, and/or (iii) fixing of the textile to the tissue comprises fixing the textile to the tissue along a width of the textile such that the tether is disposed within the gap between and/or first and second groups of barbs during the fixing.
Example 71. The method according to any one of examples 49-70, wherein an end of the tether extends outside of the subject, and/or wherein the tensioning comprises pulling on the end of the tether outside of the subject.
Example 72. The method according to any one of examples 49-71, further comprising advancing a frame into the heart of the real or simulated subject, and/or wherein contacting the textile with the tissue comprises placing the textile on the frame, such that the textile is held in position relative to and/or in contact with the tissue.
Example 73. The method according to any one of examples 49-72, wherein the textile has an implant attached thereto via the tether, the method further comprising transluminally advancing the implant into the heart of the real or simulated subject, and/or wherein fixing of the textile to the tissue results in fixing of the implant to the heart of the real or simulated subject.
Example 74. The method according to any one of examples 49-73, wherein the fixing of the textile to the tissue comprises pulling a drivebelt associated with a rotational cam having multiple barbs mounted thereon, thereby causing rotation of the rotational cam and/or causing the multiple barbs to move in a reciprocating manner through the textile and/or into the tissue and/or proximally out of the tissue and/or the textile.
Example 75. An apparatus for use with tissue of a real or simulated subject, the apparatus comprising: (i) a textile transluminally advanceable into the real or simulated subject, and/or (i) a felting tool. The textile comprises multiple fibers. The felting tool is transluminally advanceable into the subject, and/or comprises, at a distal end thereof, one or more barbs. While the textile contacts the tissue, the one or more barbs are adapted to fix the textile to the tissue by repeatedly: (i) moving distally through the textile and/or into the tissue to draw fibers of the textile into the tissue; and/or (ii) moving proximally out of the tissue and/or the textile, while leaving the fibers remaining within the tissue.
Example 76. The apparatus according to example 75, wherein the textile is a woven textile.
Example 77. The apparatus according to any one of examples 75-76, wherein the textile comprises a cell-growth-promoting substance.
Example 78. The apparatus according to any one of examples 75-77, wherein the textile comprises a synthetic polymer.
Example 79. The apparatus according to any one of examples 75-78, wherein the textile comprises a natural polymer.
Example 80. The apparatus according to any one of examples 75-79, wherein the textile comprises a combination of multiple different fiber types.
Example 81. The apparatus according to any one of examples 75-80, wherein the textile comprises collagen.
Example 82. The apparatus according to any one of examples 75-81, wherein the textile comprises cellulose.
Example 83. The apparatus according to any one of examples 75-82, wherein the textile comprises chitin.
Example 84. The apparatus according to any one of examples 75-83, wherein the textile comprises chitosan.
Example 85. The apparatus according to any one of examples 75-84, wherein the textile comprises silk.
Example 86. The apparatus according to any one of examples 75-85, wherein the textile comprises a hydrogel.
Example 87. The apparatus according to any one of examples 75-86, wherein the textile comprises polytetrafluoroethylene.
Example 88. The apparatus according to any one of examples 75-87, wherein the textile comprises polypropylene.
Example 89. The apparatus according to any one of examples 75-88, wherein the textile is a woven textile.
Example 90. The apparatus according to any one of examples 75-89, wherein the textile is a woven textile having a weft and/or a warp, the weft comprising a first fiber type and/or the warp comprising a second fiber type.
Example 91. The apparatus according to any one of examples 75-90, wherein the textile incorporates a radiopaque material.
Example 92. The apparatus according to any one of examples 75-91, wherein the textile is a non-woven textile.
Example 93. The apparatus according to any one of examples 75-92, wherein the textile is a felt.
Example 94. The apparatus according to any one of examples 75-93, wherein the felting tool is sterile.
Example 95. The apparatus according to any one of examples 75-94, wherein the textile is sterile.
Example 96. The apparatus according to any one of examples 75-95, wherein the felting tool is adapted to position the textile on the tissue.
Example 97. The apparatus according to any one of examples 75-96, wherein the one or more barbs of the felting tool each comprise one or more notches.
Example 98. The apparatus according to any one of examples 75-97, further comprising a first longitudinal catheter configured to be transluminally advanced toward an anatomical site of the subject, the first longitudinal catheter having a proximal part and/or a steerable distal part, and/or wherein the textile is advanceable into the heart of the real or simulated subject via the first longitudinal catheter.
Example 99. The apparatus according to example 98, wherein the felting tool is advanceable into the heart of the real or simulated subject via the first longitudinal catheter.
Example 100. The apparatus according to any one of examples 75-99, further comprising a second longitudinal catheter configured to be transluminally advanced toward an anatomical site of the subject, the second longitudinal catheter having a proximal part and/or a steerable distal part, and/or wherein the felting tool is advanceable into the heart of the real or simulated subject via the second longitudinal catheter.
Example 101. The apparatus according to any one of examples 75-100, wherein the textile and/or the felting tool are advanced into the heart of the real or simulated subject concurrently, such that as a portion of the textile comes into contact with the tissue, the one or more barbs fix the portion of the textile to the tissue.
Example 102. The apparatus according to any one of examples 75-101, wherein the entire textile comes into contact with the tissue prior to fixing of any portion of the textile to the tissue.
Example 103. The apparatus according to example 102, further comprising at least one tissue anchor adapted to be advanced into the heart of the real or simulated subject, and/or to temporarily anchor the textile to the tissue prior to fixing of the textile to the tissue by the felting tool.
Example 104. The apparatus according to any one of examples 75-103, further comprising a tether, wherein the barbs engage and/or fix to the tissue a first longitudinal segment of the textile, and/or wherein the tether extends through a second longitudinal segment of the textile, the second longitudinal segment being parallel to and/or distinct from the first longitudinal segment.
Example 105. The apparatus according to example 104, wherein the tether includes a first length of tether extending along a first lateral edge of the textile, and/or a second length of tether extending along a second lateral edge of the textile, parallel to the first length of tether, such that a longitudinal gap is formed along the textile between the first and/or second lengths of tether, and/or wherein the barbs are adapted to fix the textile to the tissue along the longitudinal gap.
Example 106. The apparatus according to example 104, further comprising a tether, wherein the tether extends along a single longitudinal length of the textile, and/or wherein the felting tool includes a first group of barbs and/or a second group of barbs, the first and/or second groups of barbs having a gap therebetween, wherein the barbs are adapted to fix the textile to the tissue along a width of the textile such that the tether is disposed within the gap between the first and/or second groups of barbs during the fixing.
Example 107. The apparatus according to any one of examples 75-106, further comprising a tether, wherein at least one end of the tether extends outside of the subject, and/or is accessible for tensioning thereof.
Example 108. The apparatus according to any one of examples 75-107, further comprising a frame, wherein the frame is adapted to hold the textile in position relative to the tissue during operation of the felting tool.
Example 109. The apparatus according to any one of examples 75-108, further comprising a tether and/or at least one implant attached to the textile via the tether, the implant being adapted to be transluminally advanced to the heart of the real or simulated subject and/or attached to the tissue by fixing of the textile to the tissue.
Example 110. The apparatus according to any one of examples 75-109, wherein each of the one or more barbs defines a longitudinal axis, the longitudinal axes of the one or more barbs being arranged in a plane, such that distal tips of the one or more barbs form a straight line.
Example 111. The apparatus according to any one of examples 75-110, wherein the one or more barbs are arranged such that distal tips thereof form a ring shape or a circle.
Example 112. The apparatus according to any one of examples 75-111, wherein: (i) the felting tool comprises a rotational cam associated with a drivebelt; and/or (ii) the multiple barbs are disposed along the rotational cam in a manner that pulling the drivebelt rotates the rotational cam, thereby sequentially pushing the barbs through the textile into the tissue as the cam rolls along the textile.
Example 113. The apparatus according to example 112, wherein each of the one or more barbs is attached to the rotational cam by a biasing element, the biasing element adapted to assist in rapid insertion and/or extraction of the one or more barbs into and/or out of the textile and/or the tissue.
Example 114. The apparatus according to example 112, wherein pulling of the drivebelt is further adapted to feed the textile onto the tissue prior to felting thereof.

1. An apparatus for use with tissue of a heart of a subject, the apparatus comprising:
a textile;
a tether extending through the textile; and
a tool transluminally advanceable into the heart of the subject, the tool comprising, at a distal end thereof, multiple barbs,
wherein, while the textile contacts the tissue, the tool is adapted to secure the textile to the tissue by repeatedly:
extending the barbs through the textile and into the tissue to draw fibers of the textile into the tissue; and
retracting the barbs out of the tissue and the textile, while leaving the fibers remaining within the tissue.

2. The apparatus according to claim 1, wherein the tool is adapted to position the textile on the tissue.

3. The apparatus according to claim 2, wherein the tool is configured to synchronously:
progressively position successive portions of the textile into contact with the tissue; and
progressively secure the successive portions of the textile to the tissue by driving the barbs through the textile and into the tissue.

4. The apparatus according to claim 1, wherein the tool is configured to progressively feed out and secure the textile to the tissue.

5. The apparatus according to claim 1, further comprising at least one tissue anchor adapted to be advanced into the heart of the subject, and to temporarily anchor the textile to the tissue prior to fixing of the textile to the tissue by the tool.

6. The apparatus according to claim 1, wherein the tether is impervious to the barbs of the tool as the barbs move distally through the textile.

7. The apparatus according to claim 1, wherein the barbs engage and fix to the tissue a first longitudinal segment of the textile, and wherein the tether extends through a second longitudinal segment of the textile, the second longitudinal segment being parallel to and distinct from the first longitudinal segment.

8. The apparatus according to claim 7, wherein the tether includes a first length of tether extending along a first lateral edge of the textile, and a second length of tether extending along a second lateral edge of the textile, parallel to the first length of tether, such that a longitudinal gap is formed along the textile between the first and second lengths of tether, and wherein the barbs are adapted to fix the textile to the tissue along the longitudinal gap.

9. The apparatus according to claim 7, wherein the tether extends along a single longitudinal length of the textile, and wherein the multiple barbs define a first group of barbs and a second group of barbs, the first and second groups of barbs having a gap therebetween, wherein the barbs are adapted to fix the textile to the tissue along a width of the textile such that the tether is disposed within the gap between the first and second groups of barbs during the fixing.

10. The apparatus according to claim 1, wherein the tether is arranged with respect to the textile, such that, once the textile has been secured to the tissue, tensioning of the tether contracts the tissue via contraction of the textile.

11. The apparatus according to claim 10, wherein:
the apparatus comprises an annuloplasty implant that comprises the textile and the tether,
the tissue is tissue of an annulus of a heart of the subject, and
tensioning of the tether contracts the annular tissue.

12. The apparatus according to claim 1, further comprising a prosthetic valve attached to the textile via the tether, the prosthetic valve being adapted to be transluminally advanced to the heart of the subject and secured at a native valve of the heart by fixing of the textile to ventricular tissue spaced from the implant such that the tether extends from the textile through a ventricle of the heart to the prosthetic valve.

13. The apparatus according to claim 1, wherein:
the tool comprises a rotational cam associated with a drivebelt; and
the multiple barbs are disposed along the rotational cam in a manner that pulling the drivebelt rotates the rotational cam, thereby sequentially pushing the barbs through the textile into the tissue as the cam rolls along the textile.

14. The apparatus according to claim 13, wherein each of the multiple barbs is attached to the rotational cam via a biasing element, the biasing element adapted to assist in rapid insertion and extraction of the barbs into and out of the textile and the tissue.

15. The apparatus according to claim 13, wherein pulling of the drivebelt feeds the textile onto the tissue prior to felting thereof.

16. The apparatus according to claim 13, wherein the drivebelt is associated with, or is, the tether extending through the textile.

17. The apparatus according to claim 13, wherein the tool is configured such that pulling of the drivebelt advances successive portions of the textile along the tissue prior to felting of the successive portions.

18. The apparatus according to claim 13, wherein the drivebelt is configured to lay the textile along the tissue by feeding the textile onto the tissue.

19. An apparatus for use with tissue of a heart of a subject, the apparatus comprising:
a textile;
a tool comprising:
a drivebelt that defines multiple barbs at a distal end of the tool; and
a catheter, adapted to transluminally advance the textile and the drivebelt into the heart while the textile is operatively coupled to the drivebelt,
wherein, within the heart, via pulling of the drivebelt, the tool is configured to synchronously:
progressively position successive portions of the textile into contact with the tissue; and
progressively secure the successive portions of the textile to the tissue by driving the barbs through the textile and into the tissue.

20. Apparatus, for use with tissue of a heart of a subject, the apparatus comprising:
an elongate textile; and
a tool in which the textile is loaded, the tool configured to transluminally advance the textile to the heart, and comprising, multiple barbs at a distal part of the tool, wherein the tool is configured to mechanically synchronize (a) feeding of the textile out of the tool and onto the tissue with (b) driving of the barbs through the textile and into the tissue.
